(19) United States
(12) Patent Application Publication
(10) Pub. No.: US 2010/0113912 A1
(43) Pub. Date: May 6, 2010

(54) SMALL DIAMETER MRI MARKER AND RELATED METHOD

(76) Inventors: Maeghan E. Traboulsi, Boston, MA (US); Mark Broyles, Plainville, CT (US)

Correspondence Address: MCCARTER & ENGLISH, LLP HARTFORD;CITYPLACE I, 185 ASYLUM STREET, HARTFORD, CT, 06103, US

(21) Appl. No.: 12/264,185

(22) Filed: Nov. 3, 2008

(51) Int. Cl. A61B 5/055

(52) U.S. Cl. 600/414

(57) ABSTRACT

A marker comprising a hollow body filled with a magnetic resonance imaging medium is used to mark specific surface areas of concern or sources of pain on a patient's skin prior to commencing MRI imaging procedures. The hollow body is sized such that the marker appears on less than five MRI slice images, and preferably not more than two MRI slice images, thereby enabling a physician or technician to precisely locate and assess a patient's condition based on the location of the marker in the MRI images.

### FIELD OF THE INVENTION

The present invention generally relates to magnetic resonance imaging, and more particularly, to devices and methods for marking treatment areas prior to undergoing MRI procedures, and identifying marked areas in MRI images.

### BACKGROUND

Magnetic resonance imaging (MRI) was invented in the 1970s as a means for safely and efficiently locating and identifying subcutaneous or subdermal features, and the field continues to evolve. As is described in U.S. Pat. No. 3,789,832 to Damadian, MRI imaging typically involves applying an oscillating magnetic field to tissue in the presence of a constant magnetic field. After the oscillating magnetic field is removed, spin-lattice relaxation times and spin-spin relaxation times are measured for the various nuclei in the tissue as they return to equilibrium states. By comparing the relaxation times observed in the tissue under examination with known relaxation times for normal tissues and for damaged tissues, the presence and degree of subcutaneous or subdermal damage or other injury may be detected.

The observed relaxation times may be processed and converted into a series of two-dimensional images of the three-dimensional tissue under examination, using a Fourier transform. Like a loaf of bread, the three-dimensional tissue is displayed in a series of two-dimensional images, known as “slices”, which depict the composition of the tissue beneath the surface at selected intervals for the entire width of the tissue. A typical slice may be only a few millimeters wide, thus enabling MRI machines to provide accurate images of subcutaneous or subdermal features with great precision.

However, because a typical MRI slice is so narrow, it is often difficult to pinpoint where a specific portion of injured tissue is located because of the number of slices involved. For example, in areas such as the hands, the wrists or the feet, where compact interactions between bones, muscles and tendons can occupy only a few millimeters of width, it may be difficult to locate injured or damaged tissues and precise areas of pain in limited fields of view. When the MRI is used to assess an area of concern or source of pain based on a surface indication or sensation, such as a bruise, depression or fracture, it is desired to precisely mark the surface of the particular region where the pain or condition has been sensed before commencing MRI imaging, and to be able to identify that particular region in the resulting MRI slice images.

Those of skill in the art have attempted to pinpoint surface areas of concern in MRI imagery with limited success. For example, technicians may mark these regions, which typically exist on fingers, toes, wrists, and the like, in small fields of view by applying makeshift methods and objects such as Vitamin E capsules, cough drops or almonds, which image in respective MRI slices. Although these items can be used to identify points in MRI images, they are often too large to pinpoint precise areas of interest in a finite number of slices, and cannot, by themselves, be readily affixed to a surface area of concern or source of pain.

U.S. Pat. No. 5,427,099 to Adams, which is assigned to the assignee of the present application and incorporated herein by reference in its entirety, describes a marker for MRI imaging that is formed by sealing a pair of plastic sheets to form a cavity. The cavity is filled with a lipid such as mineral oil, vegetable oil or fish oil, and the marker is applied to a patient's body prior to commencing MRI imaging. The markers are approximately two-and-one-half inches by one inch (approximately 63.5 millimeters by 25.4 millimeters) in area, and the plastic sheets are sealed with seams that are approximately one-quarter to three-quarters inches (approximately 6.35 millimeters to 19.05 millimeters) wide. Although the marker disclosed in Adams is flexible and can be affixed to a patient's skin, it is too large to be applied to a specific surface area of concern or source of pain, and cannot be effectively isolated and identified in a minimal number of resulting MRI slices.

FIGS. 1A and 1B include a series of twelve MRI slice images, numbered 1 to 12 therein, of a tissue specimen marked with a prior art MRI marker. As shown beginning in FIG. 1A, the mark created by the prior art MRI marker appears faintly at first in image 2, then grows into a large, solid mark as shown in images 4 through 11 of FIGS. 1A and 1B, before finally fading in image 12 of FIG. 1B. As can be seen, a significant drawback of such prior art markers is that the image of the marker appears in 11 imaging slices, thus substantially preventing the marker from identifying with any reasonable degree of precision a patient's point of pain or other area of interest. Yet another drawback of such prior art MRI markers is that the relatively large sizes and configurations of such markers can distort adjacent pathology.

Accordingly, it is an object of the present invention to overcome one or more of the above-described drawbacks and/or disadvantages of the prior art.

### SUMMARY OF THE INVENTION

In accordance with a first aspect, the present invention is directed to a magnetic resonance imaging marker, comprising a body, and a magnetic resonance imaging medium received within the body. The magnetic resonance imaging medium defines a maximum dimension of less than about eight mm such that the magnetic resonance imaging medium cannot visually appear on more than about five consecutive magnetic resonance imaging slices for precisely identifying a point of pain or other specifically identifiable area of interest with the marker on a magnetic resonance image thereof. The marker further includes a mounting member supporting thereon the body and including an adhesive on a surface thereof for adhesively attaching the mounting member and marker on a person's skin at the point of pain or other specifically identifiable area of interest.

Preferably, the mounting member flexibly conforms to the tissue, and the body is sufficiently small, to prevent distortion of pathology. In some embodiments of the invention, the maximum dimension of the magnetic resonance imaging medium is less than or equal to about six mm. In some embodiments of the present invention, the maximum dimension is selected so that the magnetic resonance imaging medium cannot visually appear on more than about three consecutive magnetic resonance imaging slices for precisely identifying the point of pain or other specifically identifiable area of interest with the marker on a magnetic resonance image thereof. In some such embodiments, the maximum dimension is selected so that the magnetic resonance imaging medium cannot visually appear on more than about two consecutive magnetic resonance imaging slices.

In some embodiments of the present invention, the body defines a chamber, and the magnetic resonance imaging medium is received within the chamber. In some such embodiments, the chamber is substantially spherical shaped, and the maximum dimension of the chamber and magnetic resonance imaging medium is the diameter of the chamber. In some embodiments, the magnetic resonance imaging medium is a liquid that is hermetically sealed within the chamber. In some embodiments, the shape of the body is substantially spherical, substantially cubic, substantially rectangular, substantially pyramidal, substantially conic, substantially polyhedronic, or substantially cylindrical. Accordingly, the maximum dimension can be the diameter, width, length or thickness of the magnetic resonance imaging medium. Preferably, the mounting member is a flexible, adhesive-backed substrate.

Some embodiments of the present invention are directed to a device including a plurality of markers that are axially spaced relative to each other on a releasable backing, wherein each mounting member is releasably attached to the releasable backing. In some such embodiments, each mounting member includes a portion that is not adhesively attached to the releasable backing to facilitate manual gripping of such portion and removal of the mounting member from the releasable backing. In some such embodiments, the releasable backing with plural markers mounted thereon is in roll form.

In accordance with another aspect, the present invention is directed to a magnetic resonance imaging marker comprising first means for appearing on a magnetic resonance image, for defining a maximum dimension of less than about eight mm for preventing the magnetic resonance image of the first means from appearing on more than about five consecutive magnetic resonance imaging slices, and for precisely identifying a point of pain or other specifically identifiable area of interest on a magnetic resonance image thereof. The marker further includes second means for supporting thereon the first means and for releasably attaching the first means to a person's skin at the point of pain or other specifically identifiable area of interest.

In some embodiments of the present invention, the first means is a body defining a chamber and a magnetic resonance imaging medium received within the chamber; and the second means is a mounting member supporting the body and including an adhesive thereon for adhesively attaching the mounting member to a person's skin.

In accordance with another aspect, the present invention is directed to a method of magnetic resonance imaging and precisely identifying a point of pain or other specifically identifiable area of interest on a subject, comprising the following steps:

providing a magnetic resonance imaging marker comprising a magnetic resonance imaging medium that defines a maximum dimension of less than about eight mm, and a mounting member including an adhesive on a surface thereof;

adhesively attaching the mounting member and marker on the subject, such as on the subject's tissue or a person's skin, at the point of pain or other specifically identifiable area of interest;

imaging a plurality of consecutive magnetic resonance imaging slices through the marker moving from approximately one side of the marker to approximately another side of the marker, and through subject tissue adjacent to the marker, wherein the total consecutive imaging slices through the marker is not more than about five;

recording the series of consecutive imaging slices; and

precisely identifying the point of pain or other specifically identifiable area of interest in the subject based on the consecutive imaging slices of the marker.

Some embodiments of the present invention include recording the series of consecutive imaging slices such that the resulting series of consecutive imaging slices includes no more than about two consecutive imaging slices of the marker. The method preferably further comprises adhesively attaching the mounting member and marker on the subject substantially without distorting adjacent tissue or pathology, and substantially preventing distortion of tissue or pathology by the marker throughout the imaging procedure.

One advantage of the MRI marker and method of the present invention is that they provide technologists and interpreting physicians with the ability to position a marker precisely on a patient's point of pain or a specifically identifiable point of interest. Yet another advantage of the MRI marker and method of the present invention is that communication between patients, technologists and interpreting physicians can be streamlined as the area of interest can be identified with precision.

These and other advantages of the present invention, and/or of the currently preferred embodiments thereof, will become more readily apparent in view of the following detailed description of the currently preferred embodiments and the accompanying drawings.

### BRIEF DESCRIPTION OF THE DRAWINGS

FIGS. 1A and 1B are a series of twelve consecutive MRI imaging slices of a prior art MRI marker mounted on tissue;

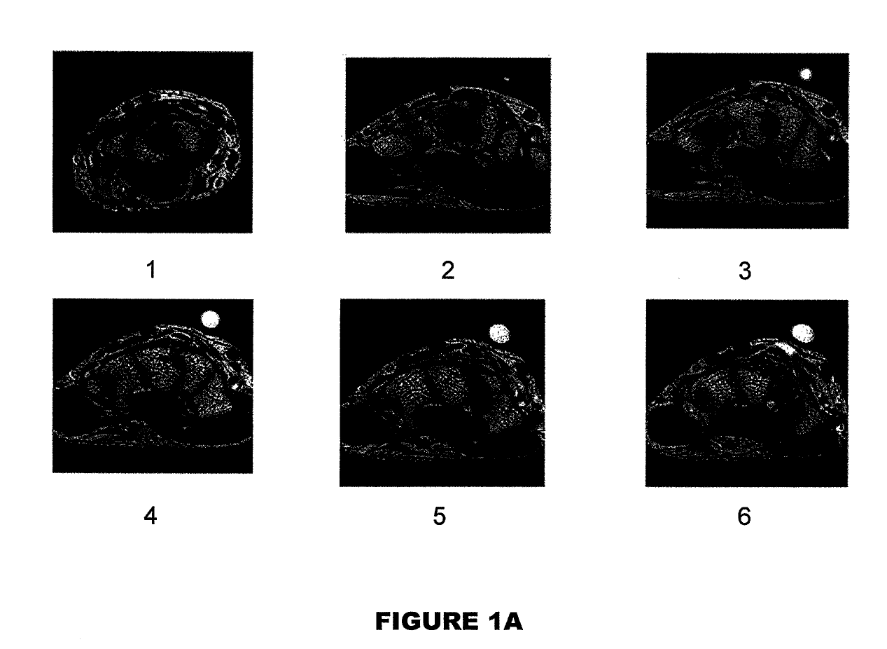

FIG. 2 is a front elevational view of an embodiment of a MRI marker of the present invention;

FIG. 3 is a side elevational view of the marker of FIG. 2;

FIG. 4 is an upper perspective view of the marker of FIG. 2;

FIG. 5 is a top plan view of a plurality of markers of FIG. 2 mounted to a carrier substrate; and

FIGS. 6A and 6B are a series of twelve consecutive MRI imaging slices of a marker of the type illustrated in FIG. 2 mounted on tissue and illustrating the manner in which the marker of the currently preferred embodiment is visible on no more than about 2 consecutive MRI imaging slices to, in turn, enable precise identification of a point of pain or other specifically identifiable area of interest.

### DETAILED DESCRIPTION OF PREFERRED EMBODIMENTS

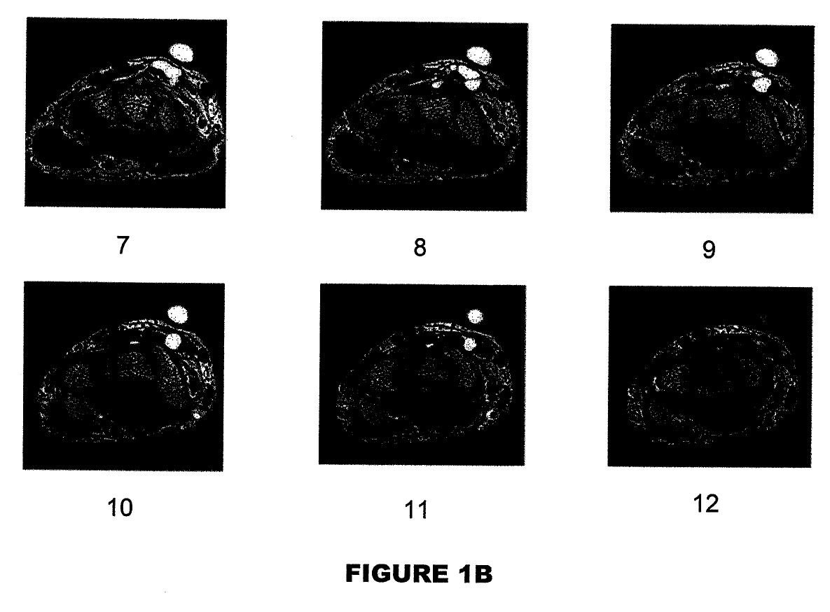

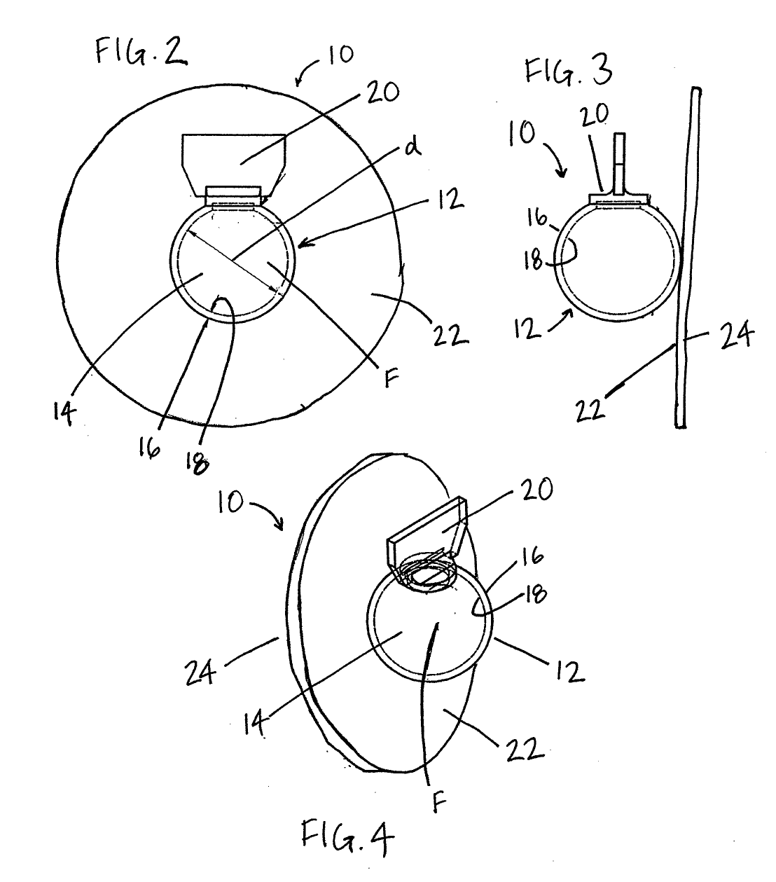

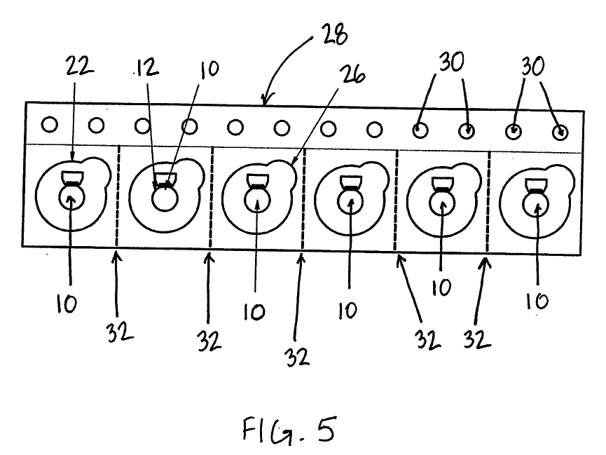

In FIGS. 2 through 5, the MRI marker herein described is indicated generally by the reference numeral 10. The marker 10 is particularly appropriate for application to a specific area of a person's skin prior to commencing MRI imaging procedures on that person.

As is shown in FIG. 2, the marker 10 comprises a body 12, the body 12 defining an interior chamber 14, an exterior surface 16 and an interior surface 18. The interior chamber 14 further defines an internal width or diameter d, and is preferably hermetically sealed or watertight. The interior chamber 14 is substantially filled with a fluid F defining a magnetic resonance imaging medium. The marker 10 is sized such that the maximum internal width d (or other maximum dimension of the magnetic resonance imaging medium F) is not more than about eight millimeters (mm), and preferably is no more than about six mm. The marker further comprises a wall portion 20 that is hermetically sealed to the body 12 after filling the internal chamber 14 with the magnetic resonance imaging medium F. As may be recognized by those of ordinary skill in the pertinent art based on the teachings herein, the marker may be filled with the magnetic resonance imaging medium in any of numerous different ways, and the magnetic resonance imaging medium may take any of numerous different configurations, that are currently known, or that later become known.

The material forming the body 12 of the marker 10 is selected such that when the body 12 is filled with the fluid F, the marker 10 is sufficiently firm to maintain its shape throughout an MRI imaging procedure. The exterior surface 16 preferably remains free of depressions, concaves, cavities, pock marks or other deformations to its ordinary shape. The body 12 is preferably formed of materials that are non-metallic to avoid adversely impacting the MRI images, and any portions of the marker that contact a person's skin, preferably are also hypoallergenic to avoid any skin irritation. Suitable materials may include low density polyethylene (LDPE). Such materials need not themselves form a magnetic resonance imaging medium, because the fluid F, and not the materials, is designed to appear in MRI slice images. As may be recognized by those of ordinary skill in the pertinent art based on the teachings herein, the body and MRI imaging medium may be formed of any of numerous different materials that are currently known, or that later become known, for performing the functions of these elements as described herein.

The MRI imaging medium F (which also may be referred to as MRI imaging contrast medium) contained within the interior chamber 14 may be any of numerous different types of MRI imaging mediums that are currently known, or that later become known, for purposes of providing an MRI image that visibly contrasts with the surrounding portions of the image, and that preferably provides a vivid MRI image of the medium. Accordingly, the MRI imaging medium should have a reasonably acceptable signal intensity based on the type of MRI image generated, and preferably has a medium signal intensity, and most preferably has a high signal intensity. Typically, the higher the signal intensity the better. Suitable magnetic resonance imaging mediums for use in the marker 10 include the solution sold by Beekley Corporation under the Radiance® brand. However, any solution, fluid or other MRI imaging medium, such as a suitable variant of the Radiance® fluid, any of the lipids disclosed in U.S. Pat. No. 5,427,099 to Adams, or others, may be used as the MRI imaging medium F. The body 12 should be filled as full of the fluid F or other magnetic resonance imaging medium as is reasonably possible and sealed watertight, and the amount of air or bubbles within the body 12 should be minimized in order to maximize the vividness of the MRI image of the medium F and of the marker 10. Preferably, the maximum width of any bubble within the body 12 is no more than about a third of the maximum internal width d of the body 12.

As described above, the currently preferred embodiments of the marker 10 feature compact geometries which retain their form and shape throughout the MRI imaging process. Preferably, the marker is free of depressions, concavities or other features that would negatively impact or cause a distraction in the resulting MRI image.

As indicated above, a significant advantage of the marker 10 is that it appears in only a limited number of consecutive MRI slices (preferably no more than about five, more preferably no more than about three, and most preferably no more than about two). This feature enables the marker 10 to be precisely applied to an area of concern or source of pain prior to commencing the MRI imaging process, and to mark that area with pinpoint accuracy in the resulting MRI image slices. Because the typical MRI slice is only a few millimeters wide, the marker 10 and, in particular, the maximum internal width d of the body 12, is sized such that the marker 10 appears in only a limited number of MRI slice images as indicated above.

As shown in FIGS. 2 through 5, a flexible, adhesive-backed substrate 22 is mounted to an area on the exterior surface 16 of the body 12. An adhesive 24 is applied to the side of the substrate 22 opposite the body 12, and the substrate is preferably sufficiently flexible to conform to the tissue substantially without distorting pathology. An example of a suitable commercially available material for use as the substrate 20 is 3M 9865a polyurethane available from Minnesota Mining and Manufacturing Co., which is thin, hypoallergenic, fluid resistant and conformable to various anatomical surfaces. However, as may be recognized by those of ordinary skill in the pertinent art based on the teachings herein, the substrate may take the form of any of numerous different materials or products that are currently known, or that later become known, for performing the function of the substrate 22 described herein.

The substrate 22 is preferably substantially planar but also flexible, and may be of any shape. Although FIGS. 2 through 5 show a plurality of substantially spherical bodies 12 mounted to a substantially round substrate 22, the shape of the substrate 22 need not correspond to the shape of the body 12 in any way. Further, the substrate 22 may be suitable for printing, so as to allow information to be communicated thereon. For example, graphics, words of encouragement, product identification or other information may be provided on the substrate 22, to comfort the person (e.g., a child) who is undergoing the procedure, to provide instructions to the technician, or for any other purpose.

When an adhesive 24 is superimposed upon the opposing side of the substrate 22, the marker 10 may be affixed to a selected area of concern or source of pain prior to commencing the MRI imaging process, and the marker 10 appears vividly on the resulting MRI slice images. The substrate 22 and adhesive 24 are preferably thin, such that the fluid-filled body 12 is maintained as close to the person's skin as possible. The adhesive 24 applied to the substrate 22 can be any conventional adhesive typically used in skin-contacting applications. Exemplary adhesives are disclosed in U.S. Pat. Nos. Re. 24,906, 3,389,827, 4,112,213, 4,310,509, 4,323,557, and 4,737,410. Such adhesives preferably transmit moisture vapor at a rate greater than or equal to that of human skin.

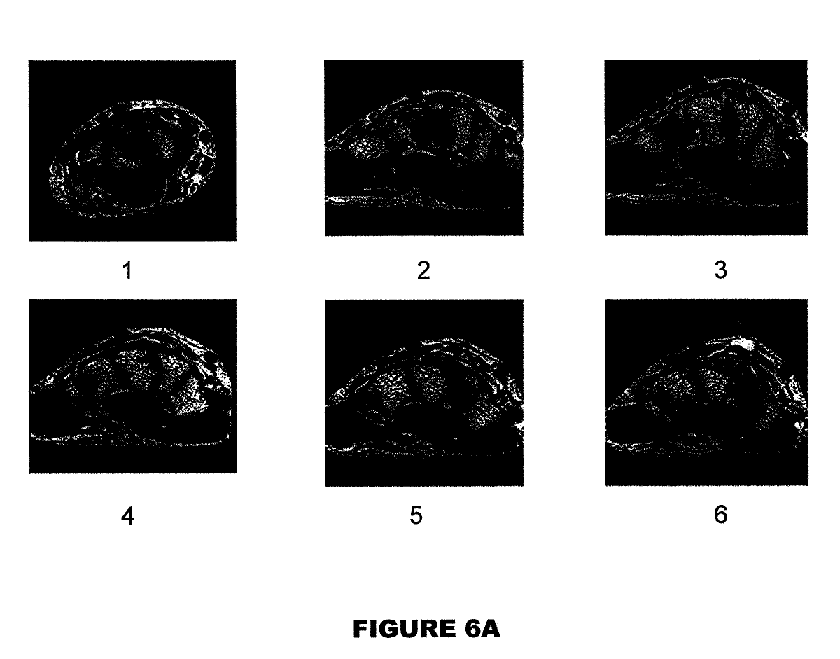

As shown in FIG. 5, a plurality of markers 10 may be mounted on a releasable backing or carrier substrate 28 that may be provided, if desired, in roll form. The backing material may be made of paper-based products, polypropylene, polyethylene, polyester or any combination of these materials, and provides for easier transportation and storage when the markers 10 are not in use. As such, a single marker 10 may be applied to a single carrier substrate 28, or one or more markers 10 may be applied to the same carrier substrate 28, which may then be stored or transported in a sheet-like form, rolled or otherwise maintained for ready dispensation and use. Preferably, the carrier substrate 28 is a continuous substrate defining a releasable surface thereon, with a plurality of markers 10 axially spaced relative to each other on the releasable surface of the carrier substrate 28. More preferably, the markers 10 define a predetermined peripheral shape and are sufficiently small, such that a user may manually grip and remove a marker 10 from the carrier substrate 28, and manually apply the marker 10 to a person's skin. Further, the sheet-like form may enable mass production of markers 10 through, for example, mechanical processes which apply the markers 10 to a continuous carrier substrate 28 in a series of operations, or all at once. As shown in FIG. 5, the continuous carrier substrate 28 is lined along one or both margins thereof with axially-spaced holes 30, which enable feeding the continuous carrier substrate 28 during production of the markers 10, and perforations 32, which enable a user to tear away or remove a portion of the continuous carrier substrate 28 corresponding to one or more markers 10, in preparation for use.

As is also shown in FIG. 5, the substrate 22 may further include a tab 26 or other extension that is not lined by the adhesive 24. Because the tab 26 is not lined by the adhesive 24, the tab 26 facilitates the removal of the marker 10 from the substrate 22 by a user before the MRI imaging procedure has begun, the placement of a marker 10 on the person's skin, and the removal of the marker 10 from the person's skin once the MRI imaging procedure is complete. The characteristics of the tab 26 may be selected based on any number of factors, such as the size of the marker 10 or the location on the person's skin to which it is to be applied. The substrate 22 should be sufficiently flexible, and the body 12 sufficiently small (as defined above) in order to allow the marker to be adhesively and conformably attached to a person's skin substantially without distorting pathology. The tab 26 may be formed of the same material as the remainder of the substrate 22, or may be formed of a different material.

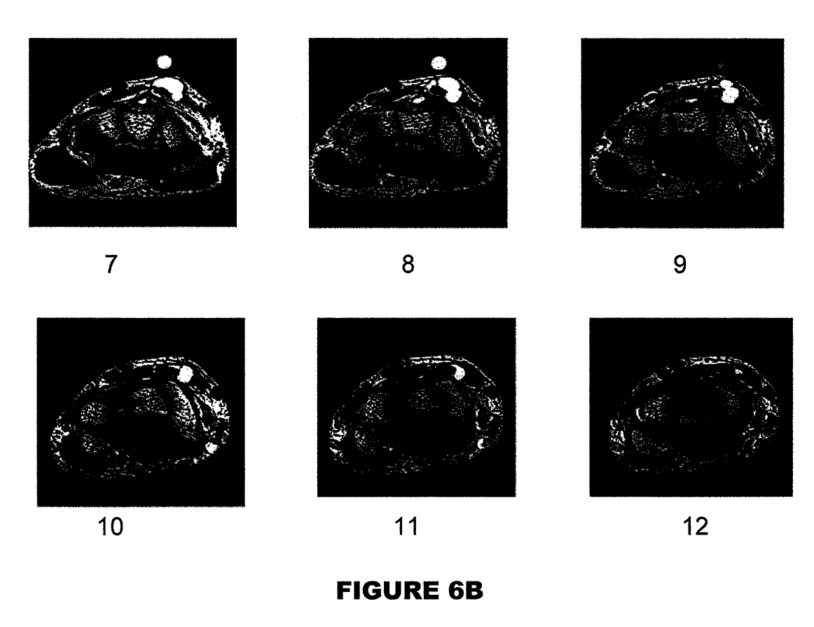

As shown in FIGS. 6A and 6B, a significant advantage of the marker of the present invention is that it appears on no more than about five consecutive MRI image slices, preferably no more than about three consecutive image slices, and most preferably no more than about two consecutive image slices. FIGS. 6A and 6B include a series of twelve consecutive MRI slice images, numbered 1 to 12 therein, of a marker 10 mounted on tissue. As can be seen, the marker 10 appears substantially in only about two consecutive MRI imaging slices (i.e., images numbered 7 and 8), appears only faintly or insubstantially in the adjacent imaging slices (i.e., images numbered 5 and 9), and does not appear at all in any of the other imaging slices. Thus, a significant advantage of the marker 10 is that is appears substantially in only about two consecutive imaging slices, and moreover, is sufficiently small (as described above the maximum dimension of the chamber is no more than about 8 mm, and preferably no more than about 6 mm), and the mounting substrate is sufficiently flexible, to substantially avoid any tissue distortion (as can be seen in imaging slices numbered 7 and 8 in FIG. 6B).

As may be recognized by those of ordinary skill in the pertinent art based on the teachings herein, numerous changes and modifications may be made to the above-described and other embodiments of the present invention without departing from its scope as defined in the appended claims. For example, the body and imaging medium may be formed of any of numerous different materials that are currently known, or that later become known. The body and/or imaging medium may take any of numerous different configurations and/or shapes. In addition, the mounting portion may take any of numerous different configurations and/or shapes. Similarly, the marker may be used on any of any numerous different subjects for any of numerous different reasons, including person's or animals for medical imaging, or inanimate objects requiring relatively precise MRI imaging analysis. Accordingly, this detailed description of the currently preferred embodiments of the present invention is to be taken in an illustrative, as opposed to a limiting sense.

What is claimed is:

1. A magnetic resonance imaging marker, comprising:
a body;
a magnetic resonance imaging medium received within the body and defining a maximum dimension of less than about eight mm such that the magnetic resonance imaging medium cannot substantially visually appear on more than about five consecutive magnetic resonance imaging slices for precisely identifying a point of pain or other specifically identifiable area of interest with the marker on a magnetic resonance image thereof; and
a mounting member supporting thereon the body and including an adhesive on a surface thereof for adhesively attaching the mounting member and marker on a subject at the point of pain or other specifically identifiable area of interest.

2. A marker as defined in claim 1, wherein the mounting member flexibly conforms to the tissue.

3. A marker as defined in claim 1, wherein the maximum dimension is less than or equal to about six mm.

4. A marker as defined in claim 3, wherein the maximum dimension is such that the magnetic resonance imaging medium cannot substantially visually appear on more than about three consecutive magnetic resonance imaging slices for precisely identifying the point of pain or other specifically identifiable area of interest with the marker on a magnetic resonance image thereof.

5. A marker as defined in claim 4, wherein the maximum dimension is such that the magnetic resonance imaging medium cannot substantially visually appear on more than about two consecutive magnetic resonance imaging slices for precisely identifying the point of pain or other specifically identifiable area of interest with the marker on a magnetic resonance image thereof.

6. A marker as defined in claim 1, wherein the body defines a chamber, and the magnetic resonance imaging medium is received within the chamber.

7. A marker as defined in claim 6, wherein the chamber is substantially spherical shaped, and the maximum dimension of the chamber and magnetic resonance imaging medium is the diameter of the chamber.

8. A marker as defined in claim 6, wherein the magnetic resonance imaging medium is a liquid that is hermetically sealed within the chamber.

9. A marker as defined in claim 1, wherein the mounting member is a flexible, adhesive-backed substrate.

10. A marker as defined in claim 1, wherein the shape of the body is one of substantially spherical, substantially cubic, substantially rectangular, substantially pyramidal, substantially conic, substantially polyhedronic, and substantially cylindrical.

11. A marker as defined in claim 1, wherein the maximum dimension is one of a diameter, width, length or thickness of the magnetic resonance imaging medium.

12. A device including a plurality of markers as defined in claim 1, wherein the plurality of markers are axially spaced relative to each other on a releasable backing, and each mounting member is releasably attached to the releasable backing.

13. A device as defined in claim 12, wherein each mounting member includes a portion that is not adhesively attached to the releasable backing to facilitate manual gripping of such portion and removal of the mounting member from the releasable backing.

14. A magnetic resonance imaging marker, comprising:
first means for appearing on a magnetic resonance image, for defining a maximum dimension of less than about eight mm for preventing the magnetic resonance image of the first means from substantially appearing on more than about five consecutive magnetic resonance imaging slices, and for precisely identifying a point of pain or other specifically identifiable area of interest on a magnetic resonance image thereof; and
second means for supporting thereon the first means and for releasably attaching the first means to a subject at the point of pain or other specifically identifiable area of interest.

15. A marker as defined in claim 14, wherein the first means is a body defining a chamber and a magnetic resonance imaging medium received within the chamber; and the second means is a mounting member supporting the body and including an adhesive thereon for adhesively attaching the mounting member to a subject.

16. A marker as defined in claim 14, wherein the maximum dimension is less than or equal to about six mm.

17. A marker as defined in claim 16, wherein the first means cannot substantially visually appear on more than about two consecutive magnetic resonance imaging slices for precisely identifying the point of pain or other specifically identifiable area of interest on a magnetic resonance image thereof.

18. A method of magnetic resonance imaging and precisely identifying a point of pain or other specifically identifiable area of interest on a subject, comprising the following steps:
(i) providing a magnetic resonance imaging marker comprising a magnetic resonance imaging medium that defines a maximum dimension of less than about eight mm, and a mounting member including an adhesive on a surface thereof;
(ii) adhesively attaching the mounting member and marker on the subject at the point of pain or other specifically identifiable area of interest;
(iii) imaging a plurality of consecutive magnetic resonance imaging slices through the marker while moving across the marker and through subject tissue adjacent to the marker, wherein the total number of consecutive imaging slices through the marker is no more than about five;
(iv) recording the series of consecutive imaging slices; and
(v) precisely identifying the point of pain or other specifically identifiable area of interest in the adjacent subject tissue based on the no more than about five consecutive imaging slices of the marker.

19. A method as defined in claim 18, further comprising imaging a plurality of consecutive magnetic resonance imaging slices through the marker while moving across the marker from approximately one side of the marker to approximately another side of the marker and through subject tissue adjacent to the marker, and wherein the total number of consecutive imaging slices through the marker is no more than about three.

20. A method as defined in claim 18, wherein the total number of consecutive imaging slices through the marker is no more than about two.

21. A method as defined in claim 18, further comprising adhesively attaching the mounting member and marker on the subject substantially without distorting adjacent tissue or pathology, and substantially preventing distortion of tissue or pathology by the marker throughout the imaging procedure.
